(19) United States
(12) Patent Application Publication
(10) Pub. No.: US 2025/0057690 A1
(43) Pub. Date: Feb. 20, 2025

(54) PHASE-CHANGE METHODS FOR COOLING GLABROUS SKIN

(71) Applicant: Jacob Brian Budnitz, Yardley, PA (US)

(72) Inventor: Jacob Brian Budnitz, Yardley, PA (US)

(21) Appl. No.: 18/485,200

(22) Filed: Oct. 11, 2023

Related U.S. Application Data
(60) Provisional application No. 63/519,620, filed on Aug. 15, 2023.
(60) Provisional application No. 63/578,602, filed on Aug. 24, 2023.

(51) Int. Cl. A61F 7/02

(52) U.S. Cl. CPC A61F 7/02; A61F 2007/0036; A61F 2007/0095; A61F 2007/0215; A61F 2007/0266; A61F 2007/0282; A61F 2007/0292

(57) ABSTRACT

The device provides a portable means of lowering human core body temperature by cooling the blood that circulates through glabrous skin. The device is fundamentally comprised of a container and a non-toxic, organic, phase-change material, such as oleic acid, selected because it transitions from a solid to liquid phase between 8 and 20° C., and preferably between 10 to 15° C. This phase-change material acts as a heatsink when placed in contact with glabrous skin thereby allowing the device to absorb heat from the circulating blood. The cooled blood then circulates through the body, lowering core body temperature. The status of the phase-change can be observed, often through transparent container walls, and the device is thermally recharged as needed.

### CROSS REFERENCE TO RELATED APPLICATIONS

This application claims the priority benefit of U.S. provisional patent application 63/519,620; filed Aug. 15, 2023; and also claims the priority benefit of U.S. provisional patent application 63/578,602, filed Aug. 24, 2023; the entire contents of these applications are incorporated herein by reference.

### INVENTION BACKGROUND

### Field of the Invention

This invention is in the field of systems and methods to cool humans by applying phase-change materials to the glabrous skin of the individual's hands, feet, or other areas of the body.

### Description of the Related Art

Humans and other mammals must regulate their physiological body temperature within narrow limits. As a result, humans and other mammals have evolved to employ various anatomical and physiological adaptions for thermoregulation, such as sweating, fur, panting, heat exchange by blood flow through blood vessels underneath various skin areas (such as glabrous or “non-hairy” skin), and the like. Humans, of course, often also employ clothing and environmental control to achieve proper thermoregulation.

Previous work in methods to transfer heat through glabrous skin includes the work of Grahn and Heller, US patent application 2016/0374853, the entire contents of which are incorporated herein by reference. Grahn and Heller taught systems and methods of circulating heat-exchanging fluid through containers placed in proximity or in contact to glabrous skin, such as in proximity to the palm area of the human hand. An example of such prior art devices is shown in FIG. 1 and FIG. 2.

FIG. 1 shows a prior art device (100) with a container (101), outflow element (102), attachment element (104), sheet (105), fastener (106), and heat exchange element (111), here filled with fluid and attached to a tube (107). Grahn and Heller teach that the temperature of the fluid may be regulated by an electrical heater or cooler, such as an electrically driven thermoregulatory device.

FIG. 2 shows the Grahn and Heller device in use. Here the device is associated with glabrous skin (109), such as the hand (108) of a human user (the user's wrist is shown as (110). In this prior art scheme, the palm is kept near or in contact with the container by an attachment element (104). Other elements include attachment elements (201), (202), and (203).

Other prior art methods include Crowder, U.S. Pat. Nos. 9,089,176 and 9,370,207; and Maurice, US patent application 2023/0018294; the entire contents of these applications are also incorporated herein by reference.

However, one drawback of these methods is that electrically driven thermoregulatory devices require bulky batteries and other mechanisms (such as mechanical circulation), and thus tend to be expensive or impractical. Thus, real-world adoption of such prior art devices has been, at best, very limited.

As a result, further advances in regulating human skin temperature through glabrous skin contact with additional types of thermoregulatory devices would be desirable.

### Other Relevant Prior Art Includes

Cheung SS. Responses of the hands and feet to cold exposure. Temperature (Austin). 2015 Feb. 27; 2 (1): 105-20.
Domalski, E. S., & Hearing, E. D. (1996). Heat Capacities and Entropies of Organic Compounds in the Condensed Phase. Volume III. Journal of Physical and Chemical Reference Data, 25 (1), 390.
Suzuki, M., Ogaki, T., & Sato, K. (1985). Crystallization and transformation mechanisms of α, β- and γ-polymorphs of ultra-pure oleic acid. Journal of the American Oil Chemists' Society, 62 (11), 1600-1604.
Lima-Oliveira G, Guidi G C, Salvagno G L, Lippi G. The impact of first clenching and its maintenance during venipuncture on routine hematology testing. J Clin Lab Anal. 2017 September; 31 (5): e22108.
Grahn D A, Murray J V, Craig, H H. “Cooling via one hand improves physical performance in heat-sensitive individuals with Multiple Sclerosis: A preliminary study, BMC Neurology 2008, 8:14
Grahn D A, Cao V H, Heller HC. Heat extraction through the palm of one hand improves aerobic exercise endurance in a hot environment. J Appl Physiol. 2005; 99:972-8
Sims, S. T., Tsai, S., & Stefanick, M. L. (2012). Abstract MP016: Innovation in Exercise: Increasing Capacity of Sedentary Obese Women with Cooling. Circulation, 125 (suppl_10).

### BRIEF SUMMARY OF THE INVENTION

The invention was inspired, in part, by the insight that although the proposals of Grahn and Heller have merit, alternative methods and devices are needed for practical use in many real-world circumstances.

The invention was also inspired, in part, by the insight that although phase-change materials, such as the melting of solid ice into water, have often been used for cooling purposes, such phase-change materials are sub-optimally suited for cooling glabrous skin. Ice is extremely cold, with a typical melting temperature of around 0° C. As a result, when brought into contact with glabrous skin, water-ice tends to be very uncomfortable upon extended contact, and can even damage the user's skin. Further, the normal physiological response of glabrous skin blood vessels, when brought into contact with water-ice, is to constrict, a phenomenon known as vasoconstriction (Cheung). This normal physiological reaction in response to extreme cold is intended to help preserve core body temperature in extreme conditions. However, this normal physiological reaction may be suboptimal when excess heat removal is desired.

The invention was also inspired, in part, by the insight that what is needed is an alternate type of phase-change material that can undergo phase transitions at a temperature that is warmer than water ice to avoid triggering vasoconstriction yet at the same time capable of absorbing significant amounts of excess body heat from a human user (who will typically have a core temperature near 37° C.). For example, a desired phase-change temperature range might be in the 8 to 20° C. range or, more ideally, in the 10 to 15° C. range.

Because this alternate phase-change material will often be stored in relatively thin-walled containers, which can break, another important consideration is that the alternate phase-change material should also be non-toxic. That is, if the phase change material itself comes into contact with the user's skin, the skin should not be damaged.

The invention was also inspired, in part, by the insight that certain organic molecules, such as oleic acid, have these desirable characteristics. This disclosure thus teaches an improved, simple, and glabrous skin-cooling device that operates without complex electronics and batteries. This simple device operates with phase-change materials that typically undergo solid to-liquid phase-changes in the 10 to 15° C. range. In some embodiments, oleic acid may be used as this phase-change material.

### BRIEF DESCRIPTION OF THE DRAWINGS

FIG. 1 shows an example of a prior art cooling device.

FIG. 2 shows another example of a prior art cooling device.

FIG. 3A shows a top-view 3D line drawing of a one-chambered version of the device.

FIG. 3B shows a perspective view of the device previously shown in FIG. 3A.

FIG. 4 shows a top-view 3D drawing of a two-chambered version of the device.

FIG. 5 shows an experimental single-chamber oleic acid cooling device with an adjustable strap.

FIG. 6 shows a single-chambered oleic acid cooling device inserted inside a two-layer sleeve configured to admit the user's hand.

FIG. 7 shows the device of FIG. 6, but in this embodiment, the single-chamber oleic acid cooling device, which was inside the two-layer sleeve in FIG. 6, is here shown laying on top of the top neoprene sleeve layer for better visibility.

FIG. 8 shows the device in use. Here, the two-layer sleeve (602) affixes the single-chambered oleic acid cooling device shown in FIG. 5 and FIG. 7 (508) and to the palm of a user's arm (800).

FIG. 9A shows a perspective view of an optional freezing module or mold, which can help freeze the heatsink into a desired shape inside the single chamber oleic acid cooling device.

FIG. 9B shows a short axis plan view of the freezing module previously shown in FIG. 9A.

FIG. 9C shows a long-axis plan view of the freezing module previously shown in FIG. 9A.

FIG. 10 shows graphs of the first experimental subjects' heart rate during aerobic exercise over time, comparing an oleic acid-based cooling device with a water-based cooling device.

FIG. 11 shows graphs of a second experimental subject's heart rate during aerobic exercise over time, again comparing a single chamber oleic acid cooling device (“Cooling”) and a water-based cooling device (Control).

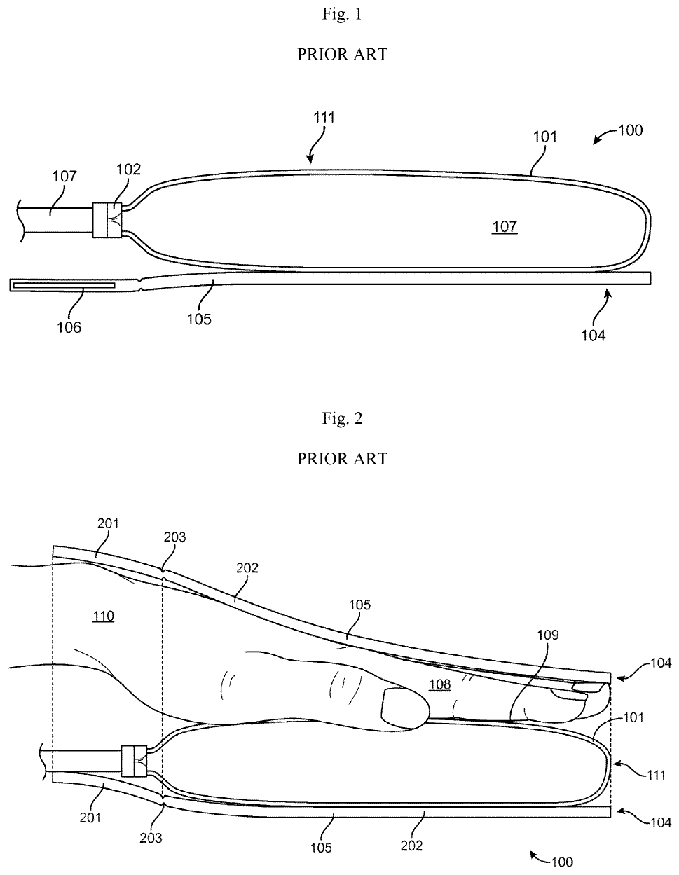

### DETAILED DESCRIPTION OF THE INVENTION

### Device Mechanics

In this disclosure, the phase-change material is often referred to in the alternative as a “heat sink” or “heat sink material.”

Phase-change material (aka “HEAT SINK”): OLEIC ACID (C₁₈H₃₄O₂)

Oleic acid has a molecular weight of 282.4614 g/mol. This is CAS registry number: 112-80-1.

According to the NIST Chemistry WebBook, SRD 69, It is alternatively referred to as: 9-Octadecenoic acid (Z)-; Δ9-cis-Oleic acid; cis-Oleic Acid; cis-9-Octadecenoic Acid; Emersol 211; Emersol 220 White Oleic Acid; Emersol 221 Low Titer White Oleic Acid; Oelsauere; Oleine 7503; Pamolyn 100; Vopcolene 27; Wecoline 00; Z-9-Octadecenoic acid; cis-Octadec-9-enoic acid; cis-Δ9-octadecenoic acid; cis-Δ9-Octadecenoate; neo-Fat 90-04; neo-Fat 92-04; Century cd fatty acid; Elaidoic acid; Emersol 210; Emersol 213; Emersol 6321; Glycon RO; Glycon WO; Groco 2; Groco 4; Groco 51; Groco 6; Hy-phi 1055; Hy-phi 1088; Hy-phi 2066; Hy-phi 2088; Hy-phi 2102; K 52; L′Acide oleique; Metaupon; Tego-oleic 130; 9-Octadecenoic acid, cis-; Elaic acid; Industrene 105; Industrene 205; Industrene 206; Oleinic acid; Pamolyn; Wochem no. 320; (Z)-9-O) ctadecanoic acid; Emersol 6313 NF; Priolene 6906; 9-(Z)-octadecenoic acid; (Z)-Octadec-9-enoic acid; 9-Octadecenoic acid (9Z)-; D) 100; Emersol 205; Extraolein 90.

The present invention leverages the properties of phase-change materials (materials that change their physical state) to optimally cool the blood that circulates through areas of glabrous skin in humans. More specifically, the device utilizes oleic acid's relatively high heat of fusion (39.6 KJ/mol) (Donalski) to absorb the heat radiated by the glabrous skin to which it is applied.

### Container: Single Compartment

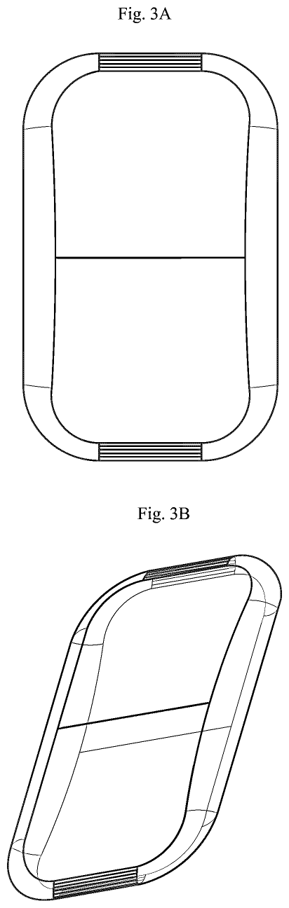

In a single-compartment (or single chamber) embodiment, as shown in FIG. 3A and FIG. 3B, the device contains only one container compartment to hold the oleic acid heat sink. The container is constructed out of thin, flexible material that does not significantly (if at all) interfere with the exchange of heat between the body and the heat sink when the device is placed in contact with glabrous skin. The flexible material allows the device to conform to the shape of the portion of the glabrous skin the user applies it to and for the user to manipulate and/or circulate the heat sink held inside. Additionally, the container is constructed to possess one or more flat, arced, or rounded surfaces large enough to at least cover the palm of an adult hand without significantly perturbing the natural positions of the palm and finger. The compartment can vary in size depending on the use case and should be sealed in some fashion to prevent the heat sink from escaping. The device could contain oleic acid in a capsule in its simplest form.

Candidate container materials include polyethylene terephthalate (PET), polyethylene (PE), silicone rubber, polyvinyl chloride (PVC), thermoplastic polyurethane (TPU), and thermoplastic elastomers (TPE) as such materials can be clear, strong, and flexible. These materials may be used individually or together and fused to encapsulate heat sink materials through ultrasonic welding, heat sealing, or other methods. The areas of the container intended to make contact with glabrous skin may be fully or in part constructed out of metal to help facilitate the transfer of heat from the glabrous skin to the heat sink material inside the device.

### Container Surfaces

Single-chambered (as well as multichambered) devices may contain curved surfaces designed to complement the natural, relaxed curvature of the hand. Such curved surfaces increase the surface area over which glabrous skin contacts the device, increasing cooling efficacy. Note: Maintained flexion (curling) or over-extension of the fingers can reduce or even halt the perfusion of blood through the hand, impeding the blood perfusion through the areas of glabrous skin. Thus, ensuring the glabrous skin being cooled remains in a natural, relaxed position is useful for optimizing the cooling effects of the device.

### Container Surface Area to Volume Ratio

The ratio of the surface area of the device's container (the area intended to contact glabrous skin) to the volume of the heat sink material can be optimized to ensure that minimal heat sink is wasted. Solid oleic acid melts at about 13° C., within the optimal cooling range. As heat is transferred from the body to the device, the heat sink material in greatest proximity to the surface of the device may warm more rapidly than more interiorly located material. This can lead to heat sink closer to glabrous skin rising to an unusable temperature before all the interior solid heatsink (which is within the useable temperature range as a solid) has melted. This “wastes” the remaining heatsink as it cannot be used to meaningfully cool the user's glabrous skin. If the surface area to volume ratio is optimized, it can be ensured that all the solid heat sink's cooling capacity can be exhausted before the liquid bilayer warms to a temperature outside of the ideal range.

Further device prototyping has shown that the surface area to volume ratio of around 1.87 cm2/mL is optimal for a device containing 205 mL of the heat sink mixture.

### Container: Multiple Compartments

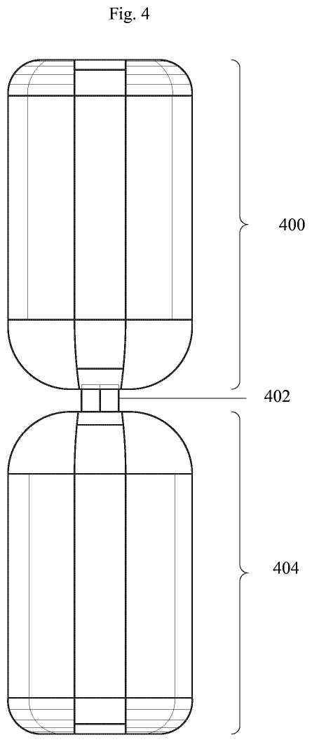

In a multiple-compartment (or multiple-chamber) embodiment, as shown in FIG. 4, the device may contain one or more compartments to hold the heat sink and/or zero, one, or multiple for the heat sink to drain into after undergoing a phase-change. Depending on the device's intended use, the compartments may or may not be connected to each other by a tube or another sealed passageway. A chamber can contain heat sink material or can be evacuated and used to collect warmed heat sink material drained from another chamber. In a preferred embodiment, the compartment or chamber is constructed from transparent, semi-transparent, or translucent material so that the phase state of the material inside the compartment/chamber can be more easily evaluated by either direct observation or by optional instrumentation.

### Cooling Compartment

A multiple-compartment form of the device contains one or more cooling compartment(s). A cooling compartment contains the heat sink in the state intended for application to glabrous skin (temperature 15° C. or less). The cooling compartment can possess at least one (or more) flat, arced, or rounded surfaces, ideally large enough to at least cover the palm of a hand. Like the single-compartment form, in the multi-compartment form, the size of these surfaces may vary depending on the use case. Since the cooling compartment is intended to hold the heatsink and serve as the site of application of the device to glabrous skin, it, like the single-compartment version, is constructed out of thin, flexible material that does not significantly or at all interfere with the exchange of heat between the body and the heatsink when the device is placed in contact with glabrous skin. The flexible material allows the compartment to better conform to the shape of the portion of the glabrous skin the user applies it to and for the user to manipulate and/or circulate the contents held inside. The cooling compartment may be connected to additional cooling and/or drainage compartments. Moreover, a cooling compartment may also act as a drainage compartment if it receives warmed, liquid heatsink runoff from another cooling compartment.

### Drainage Compartment

A multiple-compartment form of the device may have zero, one, or multiple drainage compartment(s). A drainage compartment is intended to store the heatsink after it has been warmed from a solid to a liquid, allowing melted (liquid) heatsink, such as oleic acid, from the cooling compartment to drain into the drainage compartment. The removal of warmed heat sink from the cooling compartment can prevent the formation of a bilayer in the cooling compartment that may reduce the device's cooling capabilities. The drainage compartment does not necessarily have to be constructed out of the same material as the cooling compartment, nor does it necessarily have to be constructed to fit within the same geometric constraints unless the drainage compartment and cooling compartments are meant to be used interchangeably. If interchangeable, the cooling compartment and drainage compartment should meet the material and geometric constraints described for the cooling compartments.

### Connective Passageways

Passageways (such as tubes) may be employed to connect cooling and drainage compartments. Such passageways are sealed and facilitate the movement of heatsink from one compartment to another by gravity (allowing warmed, liquid heatsink to flow down from one compartment into another), user-induced circulation, or applied pressure. Connective passageways may be continuous with the material used to create the device's compartments or constructed from a different material depending on the device's intended use.

### Optimization of Volumetric Flow Rate

Compartments may be linked by connective passageways, the dimensions of which may be modulated to control the rate of liquid heatsink flow between a device's chambers according to the Hagen-Poiseuille equation (Δp=8 μLQ/TR+), where Δp=pressure difference between two chambers, μ=dynamic viscosity, L=length of connective passageway, Q=volumetric flow rate of liquid heatsink, and R=radius of connective passageway. This equation models the volumetric flow of oleic acid heatsink between the chambers of a two-chambered device using the values characteristic of oleic acid. Specific device dimensions can be substituted for the remaining variables.

$$Δ p = \frac{8 μ LQ}{π {R}^{4}} \qquad Hagen - Poiseuille Equation$$

### Geometric Considerations

The shape of the device can be modeled after one of many smooth, 3D figures like a cylinder, sphere, or torus (donut). More involved designs may possess arced, rounded, or curved surfaces and have container shapes resembling a teardrop, egg, pill, spherical cap, chamfered rectangular prism, oblate ellipsoid, prolate spheroid, superellipsoid, or squircular cylinder as such smooth 3D figures create surfaces that complement the natural postures of the hand.

### Preparation

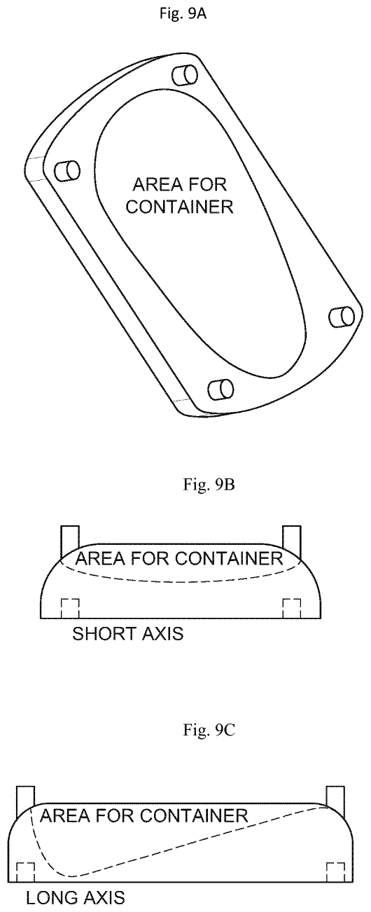

In a preferred embodiment, the device is chilled in a 5 to 10° C. environment (e.g., in a household refrigerator) until the heatsink has entirely transitioned to a “frozen” solid phase. The device may be frozen in a mold to achieve a specific shape (FIGS. 9A, 9B, and 9C). Once the heatsink is entirely solidified, the device can be removed from the cold environment. If the device is chilled in an environment colder than what the user naturally finds comfortable, it should be allowed to thaw until the device's surface temperature reaches a tolerable temperature before use.

FIGS. 9A, 9B and 9C shows an optional freezing module or mold, which can help freeze the heatsink into a desired shape inside the single chamber oleic acid cooling device. Here, one device is placed into each module, and multiple modules, each holding one device, may be stacked on top of each other and then placed in a suitable refrigerator or freezer. In some embodiments, each freezing module can have corner pegs and recesses that fit together when on top of or below another module to facilitate stacking for compact storage.

If intended to be stored for an extended time before use, the user may take the care to chill the device in a −2.2° C. environment or colder to extend the device's lifetime. After chilling to a temperature below −2.2° C., the solid structure of oleic acid crystals can change its structure upon heating while remaining solid (a polymorphic, metastable phase-change). This first-order transformation has a corresponding latent heat that decreases the rate at which the oleic acid in the heatsink material warms, ultimately increasing the time it takes for the device to reach an ideal temperature (10-15° C.). Correspondingly, the device should not be kept below −2.2° C. if intended for use in the near term as it will typically be too cold to be used comfortably or most effectively.

### Methods of Use

There are multiple ways for an individual to apply contained oleic acid to cool glabrous skin, as optimal usage methods can vary depending on container shape, material, and the state of the heatsink mixture. Namely, there are four practical procedures:

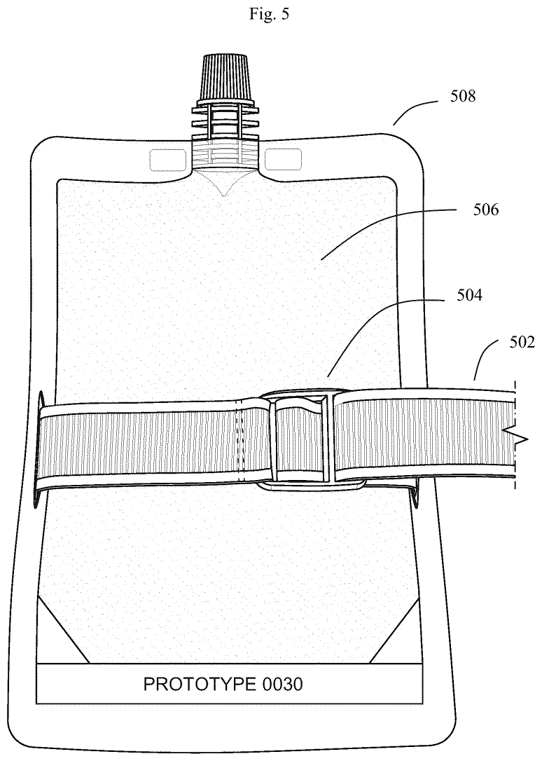

Standard One-Hand Procedure (S1HP): the S1HP involves a single device cooling a single hand. To begin, the user can position the device by resting it on a surface, suspending it from above, or balancing it on a stand. The user can then place one palm on the surface of the device to cool the hand's glabrous skin. The user can apply and remove the palm from the device as needed. The user may also periodically alternate the device face they use to cool. FIG. 5 shows that in some embodiments, a strap or other means of affixing the device to a singular hand may also be included in the device's design to allow the user's hand to move naturally instead of resting in a stationary position.

Note on Circulation: Heat is transferred from the user to the device during use, causing the heat sink material to gradually undergo a solid-to-liquid phase-change. If left uncirculated, a warmed, liquid layer of heat sink material may form around a colder interior layer, which can lead to the heat sink material warming unevenly, causing cooling capacity to vary across the device's surface. To ensure even and optimal cooling, the user can circulate the heat sink material by pressing gently on the device's surface and shifting the pressure between their wrist and fingertips using the applied hand. The user can also use their hands to break, crush, or otherwise manipulate the heat sink material once it becomes malleable. The user may also shake the device with or otherwise disturb the heat sink mixture to achieve circulation.

Standard Two-Hand Procedure (S2HP): two hands are applied to a single device in the S2HP. Like the Standard One Hand Procedure, the user applies glabrous skin of the palms to the device surface and circulates the heat sink material as needed, but in this procedure places both hands on the device simultaneously. The user can cradle the device in their hands with arms flexed at the elbow joint as if in prayer or in another comfortable position. Asymmetric hand positions can be employed as well to help the device better conform to the area to which it is applied. Depending on the shape of the device, the user can alternate their hand position across the device's surface. When used with the two-chambered device of FIG. 4, the device may be held like an upside-down kettlebell. Straps or other means of affixing the device to the hands may be included in the device's design to allow for more natural hand movement. Heat sink material may be circulated as needed. Here the cooling compartment is shown as (400), the drainage compartment is shown as (404), and the passageway between the two is shown as (402).

Crushing/Squeezing Procedure (CP): the CP can be employed once the heatsink material is warmed enough to be malleable. With one or two hands, the user can repeatedly squeeze the device by clenching and releasing their fingers. This circulates the heat sink material and may improve the device's cooling capabilities by increasing the amount of blood perfusing through the hand.

Hybrid Procedure (HP): The HP employs either the S1HP or S2HP when the heatsink mixture is too rigid or hardened to manipulate and then the SP once the heatsink mixture has sufficiently warmed. The user can alternate between the S1HP/S2HP and the SP repeatedly.

### Application of Appropriate Pressure

Glabrous skin should be pressed against the device with enough pressure to press the skin on the palm and fingers onto the device's surface without disrupting normal blood perfusion. Too much pressure can impede blood perfusion through the tissues of the hand, thereby decreasing cooling potential.

### Manufacturing Considerations

Air or other fluids not part of the heat sink material should be evacuated from the device before it is sealed, as the presence of these decreases the extent to which glabrous skin can contact the surface area of the device and thus decreases the device's cooling efficacy.

### Additional Embodiments

### Thermal Indicators

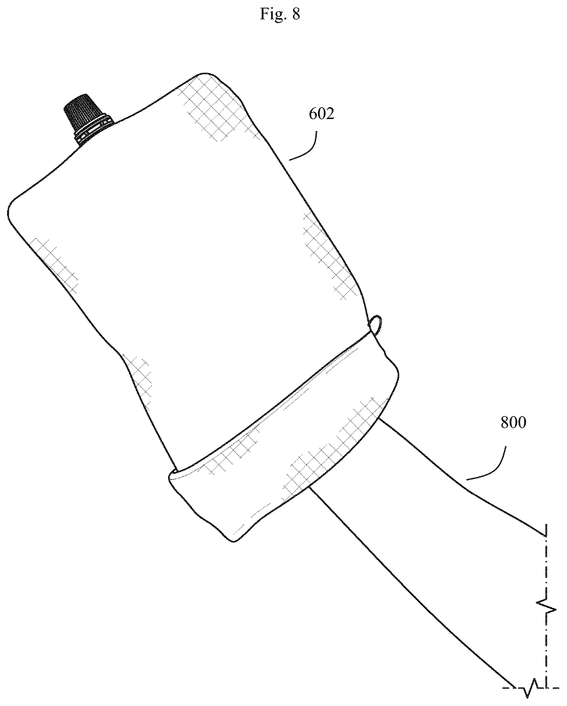

The device may contain elements that provide a visual means of assessing the temperature of the heat sink within the device. For instance, heat-sensitive (thermochromatic) pigment may be mixed into the heat sink to allow the user to visually identify when the device is too cold, too warm, or within the ideal cooling range for cooling glabrous skin. This capability is utilized by the experimental prototype in FIG. 8. The heatsink container may be designed to possess this property as well and may be constructed out of a heat-sensitive material, such as a thermochromatic plastic. A thermometer or other instrumentation may also be affixed to the container or placed within the heatsink mixture to indicate temperature.

### Dyes, Pigments, Inks & Colorants

Pigments, dyes, inks, or other additives might be mixed into the heat sink to alter its color. The container's material may be similarly altered.

Doping the Heat Sink with Other Materials

Additional materials can be added to oleic acid in the heat sink (e.g., the heat sink material is “doped”) to control the mixture's physical properties. This includes mixing or diluting oleic acid with water or another substance to augment the mixture's freezing point, change its texture, or increase its malleability when frozen. Antifreeze or additives may be included to lower the heatsink material's freezing point or to disrupt the formation of the metastable states, which typically occur upon heating to −2.2° C.ⁱⁱ

### Additional Accessories

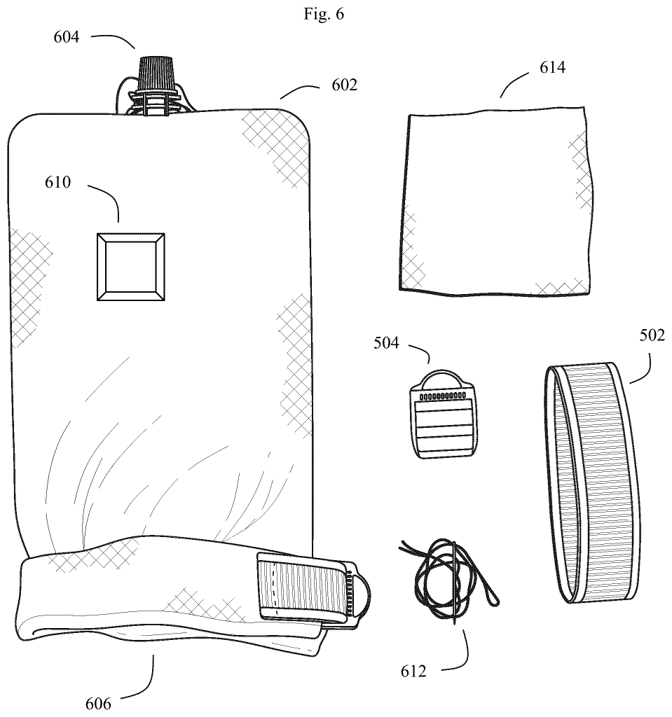

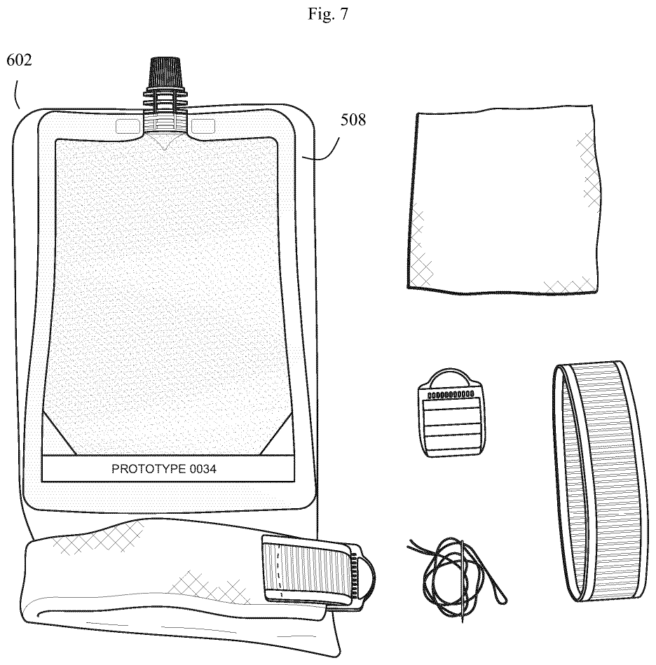

Straps, pockets, rings, grips, sleeves, or other fasteners may be affixed to the surface of the device to help keep glabrous skin in a relaxed posture or in contact with the surface of the device. Experimental embodiments of the device employing elastic straps and plastic buckles and rings can be seen in FIG. 5, FIG. 6, and FIG. 7.

In FIG. 5, note that the strap (502) can be one or more elastic bands, which may either be on top of the device or run through openings on the side. In some embodiments, these bands may be secured with a plastic fastener (504). The heat sink mixture (506) is held in a bag made of PE and PET (508). The optimal volume of phase-change material is often between 100 and 300 ml; in this embodiment, 205 mL of oleic acid is in the device.

In FIG. 6, note that the bag (508) is placed inside a two-layer sleeve (602) that is sealed on three sides (with an additional opening at the top for the oleic acid bag's cap 604). The fourth side has an opening at the bottom to admit the user's hand (606). In this embodiment, the sleeve (602) is constructed out of neoprene, but many other materials, often plastics, fabrics, and insulating materials may be used. Other materials used to configure this version of the device include an additional neoprene sleeve (614 top square), plastic fastener (504 middle), an elastic band (502 right), and an optional needle and thread (612 bottom).

In some embodiments, the sleeve may comprise either transparent material or (if the sleeve is opaque) at least one window (610) that enables observation of a phase-change status of said organic phase-change material. Here, the window may be either a square or circular window, with a diameter between about half an inch and two inches. Alternatively, optical instrumentation may be placed either inside the sleeve or employ a smaller window from outside the sleeve so that the status of the phase-change material can be determined.

Such optical instrumentation may comprise, for example, at least one photodetector and light source (such as an LED light source), as well as a battery, amplifier, analog-to-digital converter, processor, and an optional display. This optical instrumentation may be configured to detect changes in light scattering when the phase-change material transitions from a liquid to a solid phase and/or may also be configured to detect changes in color of any thermochemical material placed in said phase-change material.

Note that FIG. 7 shows the device of FIG. 6, but in this embodiment, the single-chamber oleic acid cooling device (508), which was inside the two-layer sleeve (602) in FIG. 6, is here shown lying on top of the top neoprene sleeve layer for better visibility. As shown in FIG. 8, in this configuration, when the user's hand is positioned inside the sleeve, one or more elastic bands press the oleic acid cooling device onto the glabrous skin on the user's palm. The sleeve may be elastic in other embodiments, so extra straps may be unnecessary.

As FIG. 6, FIG. 7, and FIG. 8 show, the device can also be placed inside various cases or packs or held within gloves, sleeve, or pack for insulated or discreet usage. The device or its containers may also be attached to a belt or other carrying straps.

### Potential Use Cases

Cooling glabrous skin has been shown to decrease core body temperature and lower heart rate.ⁱⁱⁱ This effect can be delivered by an oleic acid-based palmar cooling device and may be employed to provide direct benefit to athletes during training sessions, women suffering from menopausal hot flashes, patients undergoing physical rehabilitation or seeking to reduce multiple sclerosis symptoms, or any other individual wishing to lower their heart rate or core body temperature.

### Exploratory Research: Heart Rate Human Trial

### BACKGROUND

The rate at which the body's muscles produce the ATP required for muscle contraction is temperature-dependent. As internal (or core) body temperature rises above 37° C., the activity of the pyruvate kinase enzyme (an enzyme directly involved in ATP production) decreases, ultimately lowering the rate of ATP production and thereby diminishing performance during exercise or other physical activities. Measuring core body temperature directly requires placing a temperature probe into the subject's esophagus; however, heart rate has been shown to be directly proportional to core body temperature in exercising subjects, and thus measuring a subject's heart rate can provide an indirect means of determining core body temperature.^{iv} The primary objective was to determine if use of a single chamber embodiment of an oleic acid cooling device induces a significant decrease in core body temperature by measuring subjects' heart rates during continuous aerobic exercise. The secondary objective was to explore if the use of a single chamber embodiment of an oleic acid cooling device might increase exercise capacity.

### Methodology

Two subjects each performed an aerobic capacity pretest, an aerobic capacity trial with a test device, and an aerobic capacity trial with a control device. The subjects were given one day of rest between each trial and instructed not to engage in other cardiovascular activities between tests and subjects performed all three of their tests at the same time of day. Subjects were also instructed to refrain from any unnecessary physical activity between tests and agreed to not consume any caffeine, nicotine, or other stimulants within one day of any of their tests. Subject 1 performed the first aerobic capacity trial with the ice-water-based control device and the second with the oleic acid-based test device. Subject 2 performed the first aerobic capacity trial with the oleic acid test device and the second with the ice-water-based control device.

During the aerobic capacity pretest, subjects walked on the treadmill at 3.5 miles per hour (mph) and a zero-degree incline while holding the cooling devices and wearing a chest strap heart rate monitor. Subjects were also instructed not to talk or listen to music while testing as doing so may cause fluctuations in heart rate. The incline was increased by two degrees every three minutes until the subject's heart rate reached 90% of their age-adjusted maximum heart rate (age-adjusted maximum heart rate=220 beats per minute-age in years). The incline at which subjects reached their maximum heart rate was recorded.

After one day of rest, subjects performed their first aerobic trial. While wearing a chest strap heart rate monitor, subjects began walking at 3.5 mph and at an incline equivalent to 80-85% of the incline at which they achieved 90% of their maximum age-adjusted heart rate during the pretest. Once subjects reached their 70% heart rate threshold, they were given either a test or a control device to hold. (Note: beginning the test at the 70% threshold is necessary as it standardizes the subject's starting heart rate.)

The test devices were single-chambered embodiments of the cooling device with 205 mL of oleic acid mixed with a thermochromatic pigment. The test devices were initially chilled to an 8° C. surface temperature and replaced when they had exceeded a 14° C. surface temperature. The control devices were the same single-chambered embodiment but contained 205 mL of room temperature water dyed the same color as the thermochromatic pigment employed in the cooling device.

The subjects were instructed to hold the test/control device for 90 seconds and then place it down on the treadmill console for 30 seconds of rest. During the 30-second rest, the subjects were instructed to walk normally with their hands by their side. (Note: excessive hand or arm movement was seen to cause fluctuations in heart rate.) Subjects were instructed to continue walking while cyclically holding the device for 90 seconds and resting for 30 seconds. The trial was ended once their heart rate reached 90% of their age-adjusted maximum.

After another day of rest, subjects performed their second aerobic capacity trial while holding the test/control device which was not used for the first trial.

### Measures and Analysis

The subjects' heart rates were recorded each second during the pretest, cooling trial, and control trial. To determine if subjects' heart rates were significantly impacted during the aerobic excise trial using the test device compared with the aerobic exercise trial using the control device, the rate of the increase in each subject's heart rate during the aerobic excise trial using the test device was compared the rate of increase in their heart rate during the trial using the control device. A linear regression was used to calculate the slope of heart rate increase during the aerobic exercise trials using the heart rate recorded at 10 second intervals beginning when the subject's heart rate reached 70% of their pre-test maximum heart rate and ending when their heart rate reached 85% of their pre-test maximum heart rate. The fitted slopes for each subject's trial with the test device were compared to their trial with the control device using a two-sided t-test with alpha set at 0.05 for determining statistical significance.^{v}

To explore the secondary objective of determining if the use of a single chamber embodiment of an oleic acid cooling device might increase exercise capacity, the duration of time from when the subject's heart rate reached 70% of their pre-test maximum heart rate and ending when their heart rate reached 85% of their pre-test maximum heart rate was measured. The difference in duration of time was calculated for each subject's aerobic exercise trial with the test device to their aerobic exercise trial with the control device. Statistical testing was not conducted for this exploratory objective.

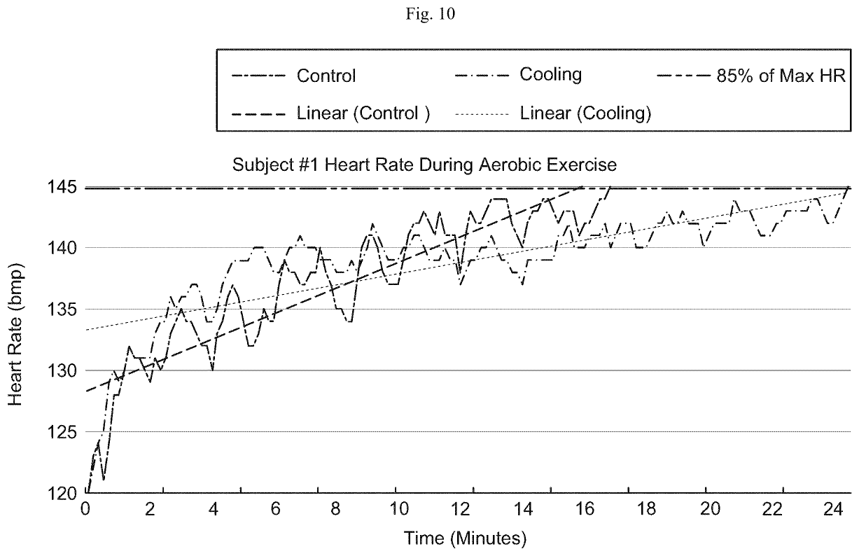

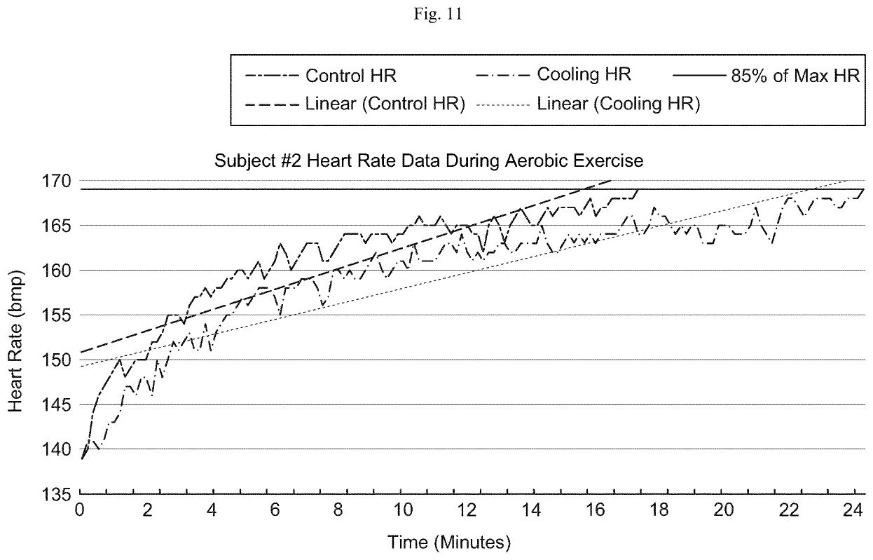

The results of these tests are shown in FIG. 10 and FIG. 11.

FIG. 10 shows a graph plotting measurements of a human subject's (Subject 1) heart rate during aerobic exercise over time, both with a single chamber oleic acid cooling device (Cooling) and a water based cooling device (Control). The graph also shows a projected slope of Subject 1's heart rate increase for both Cooling and Control conditions. The dashed line denotes 85% of Subject 1's age-adjusted maximum heart rate. This data depicts Subject 1 remaining below the 85% threshold for longer using oleic acid-based cooling.

FIG. 11 shows a graph plotting measurements of a second human subject's (Subject 2) heart rate during aerobic exercise over time, both with a single chamber oleic acid cooling device (Cooling) and a water based cooling device (Control). The graph also shows a projected slope of Subject 2's heart rate increase for both Cooling and Control conditions. The dashed line denotes 85% of Subject 2's age-adjusted maximum heart rate. This data depicts Subject 2 remaining below the 85% threshold for longer using oleic acid-based cooling.

### Findings

Both subjects tested had a significantly slower rate of heart rate increase during the aerobic trial using the test device. The rate of heart rate increase for subject 1 was +0.46 beats per minute (bmp) per minute during the aerobic trial using the test device and was +1.04 bmp per minute during the aerobic trial with the control device (p=2.9×10⁻², t-stat=11). The rate of heart rate increase for subject 2 was +0.87 bmp per minute during the aerobic trial using the test device and was +1.16 bmp per minute during the aerobic trial with the control device (p=2.8×10⁻⁵, t-stat=4.3).

Both subjects tested had an increased duration of time before reaching 85% of their maximum heart rate during the aerobic trial using the test device. Subject 1 reached 85% of their maximum heart rate in 25:00 (minutes: seconds) during the aerobic trial using the test device compared with 17:20 during the aerobic trial with the control device. Cooling increased the time Subject 1 could exercise by 44% (+7 minutes 40 seconds) (see FIG. 10). Subject 2 reached 85% of their maximum heart rate in 24:50 during the aerobic trial using the test device compared with 17:50 during the aerobic trial with the control device. Cooling increased the time Subject 2 could exercise by 39% (+7 minutes 40 seconds) (+7 minutes) (see FIG. 11).

### Why is the Oleic Acid Heatsink Giving Unexpectedly Good Results?

Without wishing to be bound by a theory, the applicant believes that the unexpectedly superior benefits of the oleic acid heatsink versus an ice-water phase transition type heatsink may be due to human physiology. It is possible that when glabrous skin contacts a very cool surface, such as ice, which often exists in the 0 to 4° C. temperature range, these cooler temperatures cause the glabrous skin blood vessels and capillaries to contract, thus diminishing the amount of body heat that can be transferred to the heatsink.

By contrast, although one might expect that oleic acid, which undergoes a solid to liquid phase change at a significantly higher temperature, might serve as a less efficient heat sink, from a physiological standpoint, this is not the case. One reason may be that the oleic acid heatsink does not cause the glabrous skin blood vessels and capillaries to contract to the same extent due to its higher temperature. As a result, even though the oleic acid heat sink is undergoing a phase transition at a higher temperature, because the glabrous skin blood vessels and capillaries are not contracted, a higher amount of blood can flow above the heat sink, resulting in a net improvement in the overall transfer of heat while exercising.

In this context, somewhat surprisingly, oleic acid was found to be an ideal candidate for a heat sink PCM (phase-change material). As previously discussed, the applicant speculates that this may be because blood perfusing through glabrous skin appears to be optimally cooled in the 5 to 15° C. range, preferably in the 10-15° C. range, and oleic acid melts at ˜13° C.^{vi} The oleic acid may be employed by the device in isolation or as a member of a mixture or a solution containing other elements.

### CONCLUSIONS

Previous research has shown that palmar cooling decreases core body temperature and increases exercise capacity. However, previous palmar cooling applications have significant portability, cost, and practicality limitations for real-world circumstances. These pilot datasets show a statistically significant slowing of subjects' heart rate increase during aerobic exercise, indicating that this invention can achieve core body temperature lowering. While more subjects would be required to demonstrate the statistical significance of improvements in the duration of aerobic capacity, the pilot data are also encouraging for this outcome.

1. A method of removing excess body heat from a human subject, said method comprising:
obtaining a device comprising at least one flexible container, wherein at least one of said flexible containers further comprises a single organic phase-change material, said organic phase-change material selected to undergo a solid to liquid phase-change at a temperature between 8 to 20° C.;
wherein said single organic phase-change material is a non-toxic organic phase-change material consisting essentially of oleic acid;
cooling said device at a temperature below said phase-change temperature until at least a majority of said organic phase-change material has transitioned to a solid phase;
placing said device directly against a glabrous skin of said human subject using only elastic force; and allowing said organic phase-change material to transition from said solid phase to a liquid phase, thus absorbing at least some excess body heat from said human subject.

2. The method of claim 1, further determining a phase-change status of said organic phase-change material, and when all or substantially all of said organic phase-change material has transitioned to said liquid phase, then removing said device from said glabrous skin of said human subject.

3. The method of claim 1, wherein said at least one said flexible container comprises at least one flexible transparent container.

4. The method of claim 3, further observing a phase-change status of said organic phase-change material through said flexible transparent container, and determining when all or substantially all of said organic phase-change material has transitioned to a liquid phase; and
when all or substantially all of said organic phase-change material has transitioned to said liquid phase, then removing said device from said glabrous skin of said human subject.

5. (canceled)

6. The method of claim 1, wherein said organic phase-change material is selected to undergo a solid to liquid phase-change at a temperature between 10 to 15° C.

7. The method of claim 1, wherein said organic phase-change material is further doped with any of a thermal indicator, colorant, or material to alter the phase-change properties of said organic phase-change material.

8. A method of removing excess body heat from a human subject, said method comprising:
obtaining a device comprising at least one transparent flexible container, wherein at least one of said flexible containers further comprises a single, non-toxic, organic phase-change material, said organic phase-change material selected to undergo a solid to liquid phase-change at a temperature between 8 to 20° C.;
wherein said organic phase-change material consists essentially of oleic acid;
cooling said device at a temperature below said phase-change temperature until at least a majority of said organic phase-change material has transitioned to a solid phase;
placing said device directly against a glabrous skin of said human subject using only elastic force; and allowing said organic phase-change material to transition from said solid phase to a liquid phase; thus absorbing at least some excess body heat from said human subject;
observing a phase-change status of said organic phase-change material through said transparent flexible container, and determining when all or substantially all of said organic phase-change material has transitioned to a liquid phase;
when all or substantially all of said organic phase-change material has transitioned to said liquid phase, then removing said device from said glabrous skin of said human subject.

9. The method of claim 8, wherein said organic phase-change material further comprises a temperature-sensitive thermal indicator;
further using said temperature-sensitive thermal indicator to determine said phase-change status of said organic phase-change material.

10. The method of claim 8, further using at least one optical window and a computerized photodetector system to determine said phase-change status of said organic phase-change material.

11. (canceled)

12. The method of claim 8, wherein said organic phase-change material is further doped with any of a thermal indicator, colorant, or material to alter the phase-change properties of said organic-phase-change material.

13. A method of removing excess body heat from a human subject, said method comprising:
obtaining a device comprising at least one transparent flexible container, wherein at least one of said flexible containers further comprises a single non-toxic, organic phase-change material, said organic phase-change material selected to undergo a solid to liquid phase-change at a temperature between 8 to 20° C.;
wherein said organic phase-change material consists essentially of oleic acid;
using a freezing module and a refrigerator to cool said device at a temperature below said phase-change temperature until at least a majority of said organic phase-change material has transitioned to a solid phase;
placing said device inside a sleeve configured to hold a human hand and placing said device directly against the glabrous skin on a palm of said human subject using only elastic force;
and allowing said organic phase-change material to transition from said solid phase to a liquid phase; thus absorbing at least some excess body heat from said human subject;
observing a phase-change status of said organic phase-change material through said transparent flexible container, and determining when all or substantially all of said organic phase-change material has transitioned to a liquid phase;
when all or substantially all of said organic phase-change material has transitioned to said liquid phase, then removing said device from said glabrous skin of said human subject.

14. The method of claim 13, wherein said freezing module is configured to be a stacking freezing module that can stack either on top of, or below, other freezing modules.

15. The method of claim 13, wherein said sleeve comprises either transparent material or at least one window that enables said observation of a phase-change status of said organic phase-change material.

16. The method of claim 13, wherein said organic phase-change material is further doped with any of a thermal indicator, colorant, or material to alter the phase-change properties of said organic-phase-change material.

17. The method of claim 13, wherein said sleeve further comprises at least one optical window, and optical instrumentation configured to use said at least one optical window to determine a phase of said organic phase-change material.
